(19) United States
(12) Patent Application Publication
(10) Pub. No.: US 2008/0124813 A1
(43) Pub. Date: May 29, 2008

(54) Bead-based radioimmunoassay

(75) Inventors: Jaw-Kang Chang, San Carlos, CA (US); Rong Ming Lyu, Foster City, CA (US)

Correspondence Address: CR MILES, P.C.;CRAIG R. MILES, 405 MASON COURT, SUITE 119, FORT COLLINS, CO, 80524, US

(73) Assignee: Phoenix Pharmaceuticals, Inc.

(21) Appl. No.: 11/985,835

(22) Filed: Nov. 17, 2007

Related U.S. Application Data
(60) Provisional application No. 60/859,762, filed on Nov. 17, 2006.

(51) Int. Cl. G01N 33/543

(52) U.S. Cl. 436/523

(57) ABSTRACT

A method of small volume bead-based radioimmunoassay which includes utilization of an immunoassay particle having a solid phase substrate linked to a target particle capture moiety which dissociably captures target particles in an immunoassay sample. Specifically, a bead linked to antibody which competitively dissociably captures target particles and labeled particles of an immunoassay sample in a small volume bead-based radioimmunoassay to allow determination of the concentration of target particles, the level of protease activity, or the level of protease inhibitor activity in such immunoassay samples.

This United States Non-provisional patent application claims the benefit of U.S. Provisional Patent Application No. 60/859,762, filed Nov. 17, 2006, hereby incorporated by reference.

### I. BACKGROUND

A method of small volume bead-based radioimmunoassay which includes utilization of an immunoassay particle having a solid phase substrate linked to a target particle capture moiety which dissociably captures target particles in an immunoassay sample. Specifically, a bead linked to antibody which competitively dissociably captures target particles and labeled particles of an immunoassay sample in a small volume bead-based radioimmunoassay to allow determination of the concentration of target particles, the level of protease activity, or the level of protease inhibitor activity in such immunoassay samples.

Radioactive elements, commonly referred to as radionuclides, are detectable because they emit energy in the form of alpha, beta or gamma rays as they decay. The use of radio-labeled proteins or peptides to trace biological activity may be one of the easiest and specific methods. The advantage of capturing radiolabeled antigen with antibody (hereinafter “radioimmunoassay”) can be that the signal of the radiolabeled antigen (whether a peptide or protein) is not affected, or only affected to a limited extent, by the physical state or chemical combination of the antibody with the radiolabeled antigen during the radioimmunoassay. This allows for the detection of a radiolabeled target particle such as a radiolabeled target antigen in complex mixture of biological particles from the radiation profile emitted.

A conventional radioimmunoassay (hereinafter “RIA”)(referring also to Example 1 below and FIG. 2) method involves detection of a specific isotope, such as I¹²⁵, isolated as part of a complex of antigen bound to a primary and a secondary antibody in solution. One example of conventional RIA is based upon the competition of an amount of I¹²⁵ labeled peptide and an amount of unlabeled peptide to dissociably bind a limited quantity of antibodies specific for the unlabeled peptide. Typically, as the quantity of unlabeled peptide (whether a standardized amount of unlabeled peptide or an unknown amount of peptide) increases, the amount of I¹²⁵-labeled particle able to bind to the limited quantity of antibodies decreases. By measuring the amount of I¹²⁵-peptide bound to such limited quantity of antibodies as a function of the concentration of a standard unlabeled peptide in solution, it is possible to construct a standard curve from which the concentration of unlabeled peptide in an amount of immunoassay sample can be determined. The conventional RIA can be used for example to determine the concentration of ghrelin peptide in human plasma and referring to FIG. 2 and Example 1, the plasma concentration intercalated from standard curves for a particular human plasma sample was about 18.8 pg per 75 μL.

Conventional enzyme immunoassay (hereinafter “EIA”) or enzyme linked immunosorbent assays (hereinafter “ELISA”) can for example utilize biotin-labeled peptide to compete with unlabeled peptide for a limited quantity of antibodies and subsequently detects bound biotin complex by reaction with streptavidin horseradish peroxidase and sequent quantitative reaction of the horseradish peroxidase with colormetric or fluorescence substrates is quite similar to RIA. However, biotinylation of peptide or protein may not be as specific as iodination of the same peptide and can affect the binding of peptide to the antibody. Also, detection of fluorescence, chemoluminescence, or colormetric regents may be impaired by a high background. Analysis using fluorescence and chemiluminescence may also require detection and analytical instrumentation which may not be available in a medical environment or if available may not readily be utilized for high throughput of immunoassay samples. In contrast, a radioactive detector useful in the detection of I¹²⁵-peptide, such as a gamma-counter, will likely be available and can be utilized for high throughput of immunoassay samples. Therefore, it remains a conventional practice to label peptides and proteins with radioactive atoms such as I¹²⁵ and employ RIA for their biological measurement. Even though conventional RIA, EIA, and ELISA have been widely practiced for many years, there remain long felt but unresolved problems with these conventional procedures.

Another significant problem with conventional RIA can be that the formation of antigen antibody complex requires a long period of time to achieve equilibrium or that the primary antibody having the required specificity or affinity to the target particle cannot be solubilized, cannot be solubilized in suitable concentration, or can only be solubilized in an amount of liquid having a sufficiently large volume to render conventional RIA unsuitable or less suitable for detection of the corresponding target particle(s).

Another significant problem with conventional RIA can that precipitation of target protein or peptide for analysis may require the use of a solubilization material which may degrade the protein or peptide (such as trichloroacetic acid) making differentiation between degradation due to proteolysis and degradation due to solubilization (such as cleavage of disulfide bonds) difficult or impossible to assess.

Another significant problem with conventional RIA may be a lack of sensitivity. Conventional RIA methods as to certain immunoassay samples cannot increase concentration of antigen sufficiently to allow capture by antibody for detection. While it is known that abnormal levels of certain low abundance proteins and peptides (such as plasma orexin A and troponin) can be an indicator or biomarker of disease, the concentration or change in concentration of these low abundance proteins and peptides in immunoassay samples cannot be measured by conventional RIA or ELISA.

Another significant problem with conventional RIA can be that certain immunoassay samples have a volume too small to be analyzed by conventional RIA methods. Certain target particles may only be obtainable in small volume immunoassay samples because the animal only produces a limited volume of the liquid containing the target particle of interest whether due to the size of the animal or because the target particle is only present in fluids collectable in small volumes from larger animals.

Another significant problem with conventional RIA can be that the determination of concentration of a target molecule takes too long a period of time. This may be due to the necessity to obtain immunoassay samples of sufficient size, the duration of time to perform the conventional assay analysis which can be several days as to certain types of RIA methods, or that a plurality of target particles may have to be quantified which may require analysis of a corresponding plurality of immunoassay samples in serial or in parallel.

Another significant problem with convention RIA can be that degradation of only one target particle can be analyzed in a single assay procedure because the detection signal is generated from a common reporting system. However, in biological fluids a plurality of target particles such as ANP, amylin, and glucagon may be coincident and degradation analysis of each coincident target particle may be desirable from the same immunoassay sample.

Additionally, there are significant problems associated with other conventional non-radioisotopic methods of assessing target particles in solution such as EIA and ELISA. Assays such as EIA and ELISA which utilize fluorescent substrates may be subject to interference from environmental factors such as pH, ionic strength, temperature, or the like. Interference from environmental factors such as these can limit the detection sensitivity and the dynamic range of these conventional assays or be misinterpreted as an inhibition of an enzyme, thus requiring extensive follow-up assays to distinguish true positives from the false positives.

Also, as to certain fluorescent assays, cleavable substrates may only be available for specific proteases. For example, in the case of measuring the activity of matrix metalloproteinases (MMPs) in solution using fluorescence resonance energy transfer assay, the fluorescent substrate is labeled with a fluorescent rodamine group on one end of a peptide and a fluorescence resonance energy donor on the other. In the absence of MMP, the quatum dot (the “QD”) emission is red light (590 nm). When the peptides are cleaved by MMP, the rhodamine groups are released, and the QD emission changes to green light (545 nm). However, fluorescent substrates for many other proteases may not yet be available.

As to each of these significant problems with conventional RIA, EIA, ELISA, or the like, the invention affords a practical solution.

### II. SUMMARY OF THE INVENTION

Accordingly, a broad object of the invention can be to provide an immunoassay particle which affords a solid substrate linked to at least one of a variety of target particle capture moieties which can be suspended in a immunoassay sample for the dissociable capture of target particles and subsequently isolated from the immunoassay sample without the conventional use of a secondary antibody and precipitation.

A second broad object of the invention can be to provide a method of immunoassay which can be performed with an amount of immunoassay sample containing target particle(s) which can be of lesser volume than utilized in performing conventional RIA, or a volume which limits or precludes the use of conventional RIA for the detection of target particles.

A third broad object of the invention can be to provide a method of immunoassay which allows detection of lesser concentration of target particles in an amount of sample solution as compared to conventional RIA, or allows detection of target particles at concentrations at which conventional RIA does not allow, or which limits or precludes the use of conventional RIA.

A fourth broad object of the invention can be to provide a method of immunoassay which allows detection of target particles in an amount of sample solution in less elapsed time as compared to conventional RIA, or allows detection of target particles in an amount of elapsed time which does not allow the use of conventional RIA.

A fifth broad object of the invention can be to provide a method of immunoassay which allows for coincident analysis of the rate of degradation of a plurality of target particles in the same sample solution. The coincident analysis of the degradation of a plurality of target particles in a single sample solution can be useful in the evaluation of the regulatory ability of a first target particle on a second target particle in the same physiological or pathophysiological condition, or the competition of the first target particle with the second target particle, or the inhibitory effect of the first target particle on the second target particle, or the like.

Naturally, further objects of the invention are disclosed throughout other areas of the specification, drawings, photographs, and claims.

### III. A BRIEF DESCRIPTION OF THE DRAWINGS

FIG. 1 shows an embodiment of the immunoassay particle linked to a target particle capture moiety for the capture of specific target particles in a particular immunoassay sample.

FIG. 2 is a plot of the concentration of human ghrelin in plasma against the amount bound to anti-ghrelin antibody utilizing a conventional RIA method.

FIG. 3 is a plot of the amount of radiolabeled ghrelin captured by anti-ghrelin antibody over elapse of an amount of time utilizing a particular embodiment of the inventive immunoassay method.

FIG. 4 is a plot of the concentration of ghrelin in plasma against the amount bound to anti-ghrelin antibody utilizing a particular embodiment of the inventive immunoassay method.

FIG. 5 is a plot of the concentration of ghrelin in plasma against the amount bound to anti-ghrelin antibody utilizing an embodiment of the inventive immunoassay method.

FIG. 6 is a plot of the concentration of obestatin in plasma against the amount bound to anti-obestation antibody utilizing an embodiment of the inventive immunoassay method as compared to a conventional RIA method.

FIG. 7 is a plot of the concentration of nesfatin in plasma against the amount bound to anti-nesfatin antibody utilizing an embodiment of the inventive immunoassay method.

FIG. 8 is a plot of the decreased concentration of radiolabeled ghrelin in solution after treatment by different amounts of pancrease enzyme as determined by an embodiment of the inventive immunoassay method.

FIG. 9 is a plot of decreasing concentration of radiolabeled ghrelin in solution upon pancreatic trypsin digestion in the presence or absence of inhibitors as determined by an embodiment of the inventive immunoassay method.

FIG. 10 is a plot of decreasing concentration of glucagon in solution over time upon isulinlysin digestion as determined by an embodiment of the inventive immunoassay method.

FIG. 11 is a plot of decreasing concentration of amylin in solution over time upon isulinlysin digestion as determined by an embodiment of the inventive immunoassay method.

FIG. 12 is a plot of decreasing concentration of glucagon and amylin in solution over time in a single sample as determined by an embodiment of the inventive immunoassay method.

### IV. DETAILED DESCRIPTION OF THE PREFERRED EMBODIMENTS

A method of small volume bead-based radioimmunoassay which includes utilization of an immunoassay particle having a solid phase substrate linked to a target particle capture moiety which dissociably captures target particles in an immunoassay sample. Specifically, a bead linked to antibody which competitively dissociably captures target particles and labeled target particles of an immunoassay sample in a small volume bead-based radioimmunoassay to allow determination of the concentration of target particles, the level of protease activity, or the level of protease inhibitor activity in such immunoassay samples.

The term “immunoassay particle” means a solid phase substrate which includes at least one type of link element such as a molecule, functional group, ion, or atom which retains a target particle capture moiety having specificity for at least one target particle. The link element typically provides a functional group(s) such as amino groups or carboxyl groups available for linking to the target particle capture moiety in the presence of a crosslinking material, for example, glutaraldehyde, carbodiimide, diazoto compounds, or other suitable crosslinking material which generates a linking bond between the solid phase substrate and the target particle capture moiety which can be a covalent bond such as an amide, ester, ether, sulfonalmide, disulfide, azo, or the like, or as to other link elements a non-covalent bond such as physical adsorption, and specifically includes, without limitation of the forgoing, silanized iron oxide magnetic particles such as BIOMAG particle available from Polyscience, Inc., 400 Valley Road, Warrington, Pa. 18976 and non-magnetic particles available from Spherotech Inc. 1840 Industrial Dr., Suite 270, Libertyville, Ill. 60048. [catalog number].

The term “solid phase substrate” means a material such as latex, polystyrene, polypropylene, polyethylene, iron oxide, magnetic iron oxide which can further include a substrate matrix comprising a plurality of pores, interstices, or the like.

The term “immunoassay sample” means an amount of sera, tissue culture medium, plasma, or biological fluid such as blood, lymph, urine, sputum, joint fluid, spinal fluid, saliva, or an amount of a liquid which contains an amount of the target particle, and with respect to the various embodiments of the inventive immunoassay can be combined or diluted with an amount of a diluent to establish a concentration of the target particle in the inventive assay detection range, or a solution containing soluble target particle, molecular analogs of the target particle, or fragments thereof.

The term “target particle” means the molecule or molecules such as protein(s), protein fragment(s), peptide(s), antibody(ies), nucleic acid(s), or regions or fragments thereof, capable of dissociable capture by the target particle capture moeity.

The term “labeled particle” means a particle such as protein(s), protein fragment(s), peptide(s), antibody(ies), nucleic acid(s), or regions or fragments thereof, capable of dissociable capture by the target particle capture moeity which further includes a non-dissociably bound radioactive material such as 125 Iodine (“I¹²⁵”).

The term “target particle capture moiety” means a molecule or molecules capable of dissociable capture of at least one target particle including, but not limited to a protein or polypeptide such as an antibody, or an antibody fragment, specific to a particular target particle which can be generated by various procedures for the production of polyclonal antibodies or monoclonal antibodies in rabbits, mice, rats, sheep, goats, horses, or the like, or produced by recombinant biological techniques.

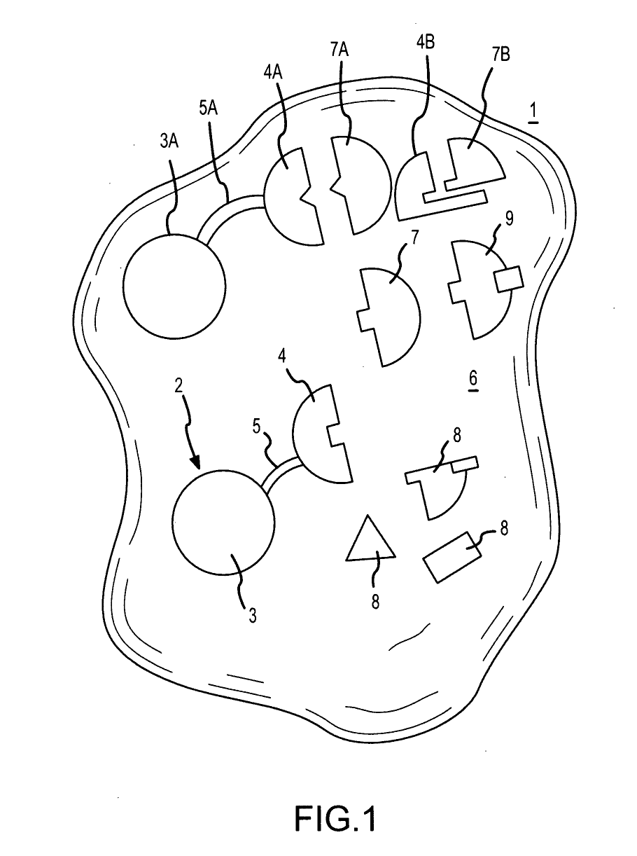

Now referring primarily to FIG. 1 and Examples 2A and 2B, the inventive solid phase immunoassay system (1) provides an immunoassay particle (2) which provides a solid phase substrate (3) to which a target particle capture moiety (4) can coupled by a link element (5). Two non-limiting examples of an immunoassay particle (2) suitable for use with the inventive solid phase immunoassay system (1) can be a silanized magnetic iron oxide particle or a polystyrene particle which provides a surface on which antibodies can be immobilized by Schiff's base linkages via glutaraldehyde. However, these specific examples or an immunoassay particle (2) are not intended to limit the definition of immunoassay particle (2), to including any particular solid phase substrate (3), link element (5), or target particle capture moiety (4) and it is intended that a numerous and wide variety of solid phase substrates (3), link elements (5) and target particle capture moieties (4) in various permutations and combinations be encompassed by the term immunoassay particle (2), and specifically with respect to the silanized iron oxide magnetic particles and non-magnetic polystyrene particles above described and utilized in the Examples below, it is not intended that these solid phase substrates (3) be limited to the specific particle configurations or distributors described but rather it is intended that the numerous and wide variety of iron oxide magnetic particle and non-magnetic particle configurations available and equivalents thereof be encompassed by the term sold phase substrate (3).

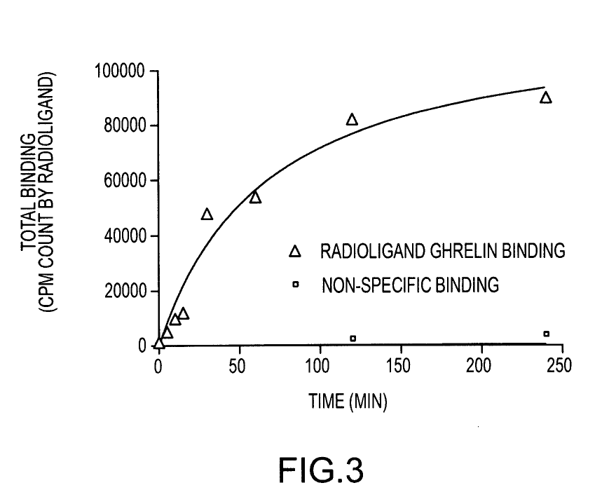

Now referring primarily to FIG. 3 and Example 3, the inventive immunoassay particle (2) can be combined with an amount of immunoassay sample (6). The amount of immunoassay sample (6) into which the immunoassay particle (2) can be entrained can be any amount which allows suspension of the immunoassay particles (2) in a manner which allows capture (typically dissociable capture) of an amount of the target particle (7) solubilized in the immunoassay sample (6). As to certain embodiments of the inventive immunoassay system (1) the amount of immunoassay sample (6) can be the amount of immunoassay sample utilized in conventional RIA procedures, or the amount of immunoassay sample which affords the sensitivity of conventional RIA procedures, or the amount which affords the sensitivity of conventional RIA procedures in a lesser total volume of immunoassay sample, and as to other embodiments of the inventive immunoassay system can be a volume of between about 25 microliters and about 35 microliters, a volume of between about 30 microliters and about 40 microliters, a volume of between about 35 microliters and about 45 microliters, a volume of between about 40 microliters and about 50 microliters, a volume of between about 45 microliters and about 55 microliters, a volume of between about 50 microliters and about 60 microliters, a volume of between about 55 microliters and about 65 microliters, or a volume of between about 60 microliters and about 70 or as to particular embodiments of the immunoassay system a volume of about 150 μL as described by Example 4, or a volume of about 60 μL as described by Example 5.

When utilizing the immunoassay particle (2) in accordance with the invention, or as part of the inventive immunoassay methods, or the inventive immunoassay methods specifically described in Examples 3-12, the immunoassay particle (2) can be utilized in a lesser volume of immunoassay sample (6) as compared to conventional RIA to dissociably bind target particles (7) such as ghrelin, obestatin, amylin, or glucagons with similar or better results as compared to conventional RIA for a variety of applications. The immunoassay particle (2) can then be incubated with the immunoassay sample (6) for a period of time typically between 2 hours and 12 hours to allow dissociable capture (equilibrium between rate at which the target particle (7) is bound to the target particle capture moiety (4) and the rate at which the target particle (7) is released from the target particle capture moiety (4)) of the target particle (7) by the target particle capture moiety (4). In a subsequent step, an amount of labeled particle (9) can be combined with the immunoassay sample (6) to allow dissociable capture of the labeled particle (6) with the target particle capture moiety (4) in competition with the amount of target particle (7) in the immunoassay sample (6). As can be understood the greater the concentration of target particle (7) in the immunoassay sample (6) the less the amount of labeled target particle (9) that will be dissociably captured by the target particle capture moiety (4).

Because the inventive immunoassay particle (2) can then be removed or separated from the immunoassay sample (6) (whether by magnetic force, centrifugal force, or filtration) without the conventional use of a secondary antibody or the use of precipitation, and washed with an amount of wash solution (such as an amount 150 mM NaCl, 100 mM sodium phosphate, 1% BSA and 1% Triton X-100) as further described below, nonspecific binding of other particles (8) in the immunoassay sample (6) to the immunoassay particle (2) can be decreased, decreased as compared to conventional RIA, or substantially eliminated. As such, the background signal generated using the immunoassay particle (2) in accordance with the inventive immunoassay methods can be correspondingly decreased, decreased as compared to conventional RIA, or substantially eliminated. The corresponding increase in the signal to noise ratio obtained using the inventive immunoassay particle (2) or inventive immunoassay methods, or both, affords a decreased period of time to complete the inventive immunoassay method. As shown for example by Examples 4, 6 and 9, the inventive immunoassay method can be completed for a sample containing nesfatin in a one day period of time and for obestatin or ghrelin in a two day period as compared to the conventional method of radioimmunoassay for ghrelin as described by Example 1 which requires 3 days to achieve substantially the same results. As such, the inventive immunoassay method can afford a decreased sample volume and a decreased immunoassay time period with the same or similar sensitivity as compared to the conventional immunoassay methods.

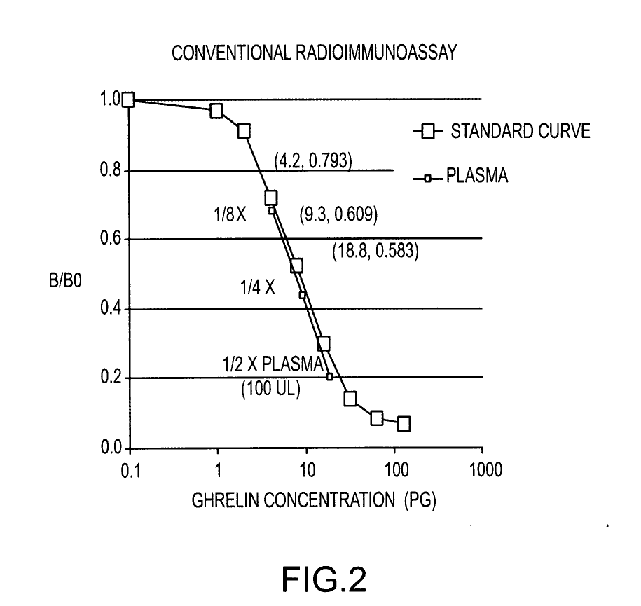

Now referring primarily to Table 1 and FIGS. 2, 4 and 5, which summarizes the results of Examples 1, 4 and 5 which compare the inventive immunoassay method which utilizes an immunoassay sample (6) having a total volume of about 150 μL (Example 5) or about 60 μL (Example 4) to a conventional RIA method which utilizes a total volume of about 300 μL (Example 1) for the assay of the human peptide ghrelin to produce a standard curve of human peptide ghrelin concentrations of about 1 picogram per mililiter (“pg/mL”), about 2 pg/mL, about 4 pg/mL, about 8 pg/mL, about 16 pg/mL, about 32 pg/mL, about 64 pg/mL, and about 128 pg/mL to determine the concentration (pg/mL) (or the amount of human peptide ghrelin) in samples of human plasma. The numerical values represent a mean with S.E. from four discrete lots of silanized magnetic iron oxide beads each measurement taken in duplicate.

TABLE 1

| | | | Example 1 |
|---|---|---|---|
| | Example 5 | Example 4 | Conventional RIA |
| | 60 μl volume | 150 μl volume | 300 μl volume |
| STD | 0.928 ± 0.17 | 0.978 ± 0.06 | 1.13 ± 0.16 |
| 1 pg/ml | | | |
| STD | 2.26 ± 0.13 | 1.79 ± 0.30 | 1.79 ± 0.12 |
| 2 pg/ml | | | |
| STD | 4.57 ± 0.32 | 4.00 ± 0.49 | 4.06 ± 0.13 |
| 4 pg/ml | | | |
| STD | 7.52 ± 0.24 | 8.43 ± 0.69 | 8.12 ± 0.13 |
| 8 pg/ml | | | |
| STD | 15.45 ± 0.79 | 16.98 ± 0.06 | 16.0 ± 0.01 |
| 16 pg/ml | | | |
| STD | 29.65 ± 2.74 | 31.49 ± 1.29 | 31.45 ± 0.60 |
| 32 pg/ml | | | |
| STD | 56.46 ± 2.7 | 68.30 ± 3.85 | 57.1 ± 4.88 |
| 64 pg/ml | | | |
| STD | 132.3 ± 12.04 | 111.3 ± 16.2 | 131.8 ± 3.36 |
| 128 pg/ml | | | |
| Human | 522.5 ± 93.1 | 381.5 ± 18.8 | 370.5 ± 6.71 |
| Plasma | | | |
| (pg/ml) | | | |

As can be understood from the results summarized in Table 1, the inventive immunoassay particle can be utilized in accordance with the methods of Examples 4 and 5 respectively to achieve results which are the substantially the same or better than utilizing the conventional RIA method with the further advantages of using a lesser volume of human plasma and performance of the assay in lesser period of time.

Now referring to Table 2, which summarizes the results using the inventive immunoassay system (1) in accordance with the procedures described in Examples 1, 4 and 5 to compare the inventive immunoassay method which utilizes an immunoassay sample (6) having a total volume of about 60 μL or about 150 μL to the conventional RIA method which utilizes a total volume of about 300 μL for the assay of the mouse peptide obestatin to produce a standard curve of mouse peptide obestatin concentrations of about 1 picogram per mililiter (“pg/mL”), about 2 pg/mL, about 4 pg/mL, about 8 pg/mL, about 16 pg/mL, about 32 pg/mL, about 64 pg/mL, and about 128 pg/mL to determine the concentration pg/mL or amount of mouse peptide obestatin in samples of mouse plasma. The numerical values represent a mean with S.E. from three discrete lots of silanized magnetic iron oxide beads each measurement taken in duplicate.

TABLE 2

| | | | Example 1 |
|---|---|---|---|
| | Example 5 | Example 4 | Conventional RIA |
| | 60 μl volume | 150 μl volume | 300 μl volume |
| STD | 1.11 ± 0.17 | 1.04 ± 0.045 | 1.59. ± 0.233 |
| 1 pg/ml | | | |
| STD | 1.87 ± 0.12 | 1.92 ± 0.06 | 2.13 ± 0.32 |
| 2 pg/ml | | | |
| STD | 3.906 ± 0.26 | 3.86 ± 0.33 | 4.43 ± 0.63 |
| 4 pg/ml | | | |
| STD | 7.92 ± 0.21 | 8.38 ± 0.26 | 7.57 ± 0.18 |
| 8 pg/ml | | | |
| STD | 15.06 ± 1.05 | 14.86 ± 0.52 | 15.7 ± 0.72 |
| 16 pg/ml | | | |
| STD | 32.34 ± 3.26 | 31.79 ± 0.24 | 33.78 ± 1.89 |
| 32 pg/ml | | | |
| STD | 67.92 ± 2.67 | 65.35 ± 7.71 | 64.21 ± 1.88 |
| 64 pg/ml | | | |
| STD | 118.5 ± 18.05 | 127.30 ± 0.50 | 124.9 ± 2.5 |
| 128 pg/ml | | | |
| Mouse | 152.6 ± 9.81 | 209.7 ± 18.2 | 243 ± 44 |
| Plasma | | | |
| (pg/ml) | | | |

Again as can be understood from the results summarized in Table 2, the immunoassay particle (2) can be utilized in accordance with the methods of Examples 4 and 5 respectively to achieve results which are the substantially the same or better than conventional RIA methods with the further advantages of using a lesser volume of immunoassay sample (6) and to perform the immunoassay method in less time.

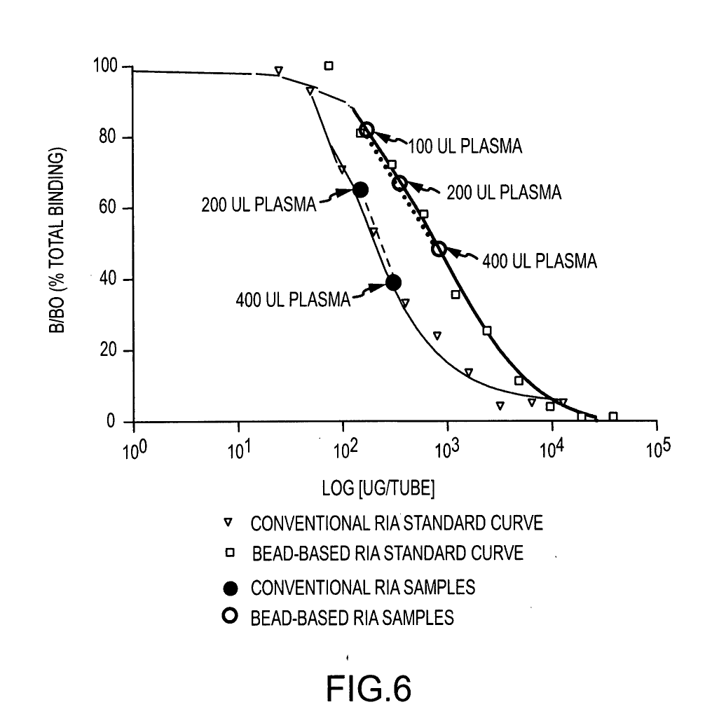

Now referring primarily FIG. 6 which provides a comparison of the results of an embodiment of the inventive immunoassay method which can be performed in accordance with Example 6 in a one day immunoassay period (in the 24 hour period subsequent to entraining the immunoassay particle (2) in an amount of immunoassay sample (6) to the determination of concentration of the target particle (7) in the immunoassay sample (6)) with the results of the conventional RIA method performed in accordance with Example 1 which requires a three day period to assay concentration of a target particle (7) in an immunoassay sample (6), such as the amount of nesfatin in rat plasma. As shown in FIG. 6, the results of the inventive immunoassay method performed in a one day period (although as to certain embodiments of the invention the immunoassay period can be even less than a one day period) are comparable to the results obtained using the conventional RIA method requiring a significantly greater period of time.

Now referring to Table 3 which provides a summary of the results of Example 7, it can be understood that the inventive immunoassay method can achieve consistent results even when the immunoassay sample (6) contains very low concentration of target particle (7). For example, when an original 20 μL immunoassay sample (6) of human saliva having a known concentration of ghrelin peptide of about 80.8 pg/mL is diluted to one half the original concentration of ghrelin peptide or one-twenty fifth the original concentration of ghrelin peptide by dilution (the original 20 μL sample brought to 40 μL or 500 μL respectively) for incubation with the immunoassay particle (2) and the subsequent incubation with the labeled particle (9), accurate determination can be made of the concentration of ghrelin peptide in the human saliva immunoassay sample (6).

TABLE 3

| | Concentration Ghrelin |
|---|---|
| Dilution Value (Total Volume) | Peptide (Picograms/mL) |
| Human saliva (20 μL) | 80.8 pg/mL ± 3.6 |
| 2:1 Dilution (40 μL) | 74.5 pg/mL ± 24 |
| 25:1 Dilution (500 μL) | 89.4 pg/mL ± 5.6 |

Similarly, and now referring to Table 4, the concentration of a target peptide (7) such as the ghrelin peptide in an immunoassay sample (6) such as human plasma can be accurately determined over a wide range of concentrations in the immunoassay sample (6). As shown by Example 8, immunoassay samples (6) having a volume of 20 μL of human plasma of known ghrelin peptide concentration of 126 pg/mL can be diluted to reduce the original immunoassay sample (6) concentration by about 3 times, about 12 times, and about 25 times respectively prior to dissociable capture of the target particle (7) and subsequent dissociable capture of the labeled particle (9) by the inventive immunoassay method. Even at very dilute concentration, determination can be made of the concentration of ghrelin peptide in the original immunoassay sample (6).

TABLE 4

| | Concentration Ghrelin |
|---|---|
| Dilution Value (Total Volume) | Peptide (Picograms/mL) |
| Human plasma (20 μL) | 126 pg/ml ± 5.3 |
| 3:1 Dilution (40 μL) | 118.1 pg/ml ± 21.8 |
| 25:1 Dilution (500 μL) | 135 pg/ml ± 22.1 |

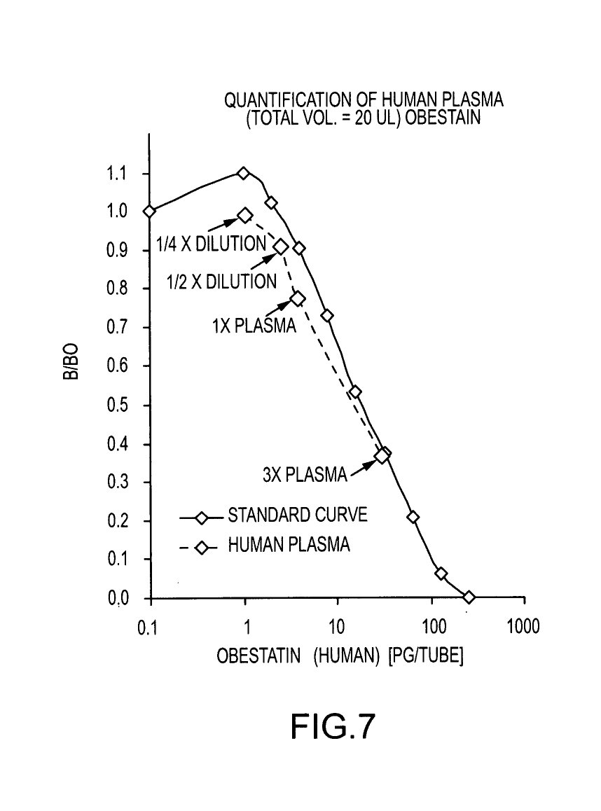

Now referring to FIG. 7 which shows that the inventive immunoassay system (1) can be utilized to determine the concentration of a target peptide (7) in an immunoassay sample (6) over a wide range of concentrations in the linear range of the standard curve. By utilizing an embodiment inventive immunoassay method as described in Example 9, determination of concentration of a target particle (7) in an immunoassay sample (6) can remain in the linear range of the standard curve over a range of concentration of target particle (7) in immunoassay sample (6) even when the concentration of a target particle (7) in an immunoassay sample (6) is close to the detection limit of the immunoassay method and even when the immunoassay sample (6) has a comparatively small volume of about 5 μl, 10 μl, 20 μl, or 100 μl.

Now referring to Table 5 and Example 10, an embodiment of the inventive immunoassay system (1) can be utilized to provide determination of a plurality of different target particles (7) in the same immunoassay sample (6)(also referred to as a “Mulitplex Assay”). For example, the embodiment of the inventive immunoassay system (1) utilized in Example 10 allows determination of concentration of orexin A, ghrelin, and obestatin in the same sample of human plasma. To achieve determination of concentration of a plurality of different target particles (7) in the same immunoassay sample (6), two different immunoassay particles (2)(see FIG. 1 which shows a first immunoassay particle (2) and a second immunoassay particle (2A)) can be provided, the first immunoassay particle (2) providing a first target particle capture moeity (4) capable of dissociable capture of a first target particle (7) and the second immunoassay particle (2A) providing a second target particle capture moeity (4A) capable of dissociable capture of a second target particle (7A). The first immunoassay particle (2) and the second immunoassay particle (2A) can further provide a first solid phase substrate (3) separable from a second solid phase substrate (3A). For example, the first solid phase substrate (3) can be a magnetic solid phase substrate or attracted by magnetic force while the second solid phase substrate (3A) can be non-magnetic or not attracted by magnetic force. Application of magnetic force to the first solid phase substrate (3) and the second solid phase substrate (3A) can allow the first solid phase substrate (3) to be separated from the second solid phase substrate (3A) as described in further detail by Example 10. The remaining immunoassay sample (6) can be assayed to determine the concentration of a third target particle (7C) by conventional RIA methods.

As further shown by the results summarized by Table 5, the Multiplex Assay can be utilized to determine the concentration of at least two target particles (7)(7A) in single volume of immunoassay sample (6) of less than 100 μl and in those embodiments of the invention utilized to further determine a concentration of a target particle (7) which includes use of a conventional RIA methods than an immunoassay sample (6) of greater volume can be used such as 150 μl or more.

TABLE 5
MULTIPLEX ASSAYS OF HUMAN SERUM.

| Target | Detected Concentration | Detected Concentration |
|---|---|---|
| Particle | 200 uL plasma) | 60 uL plasma |
| Orexin A | 23.6 ± 0.26 pg/ml | 34.5 ± 0.16 pg/ml |
| Ghrelin | 146 ± 12 pg/ml | 139 ± 22 pg/ml |
| Obestatin | 148 ± 1 pg/ml | 168 ± 24 pg/ml |

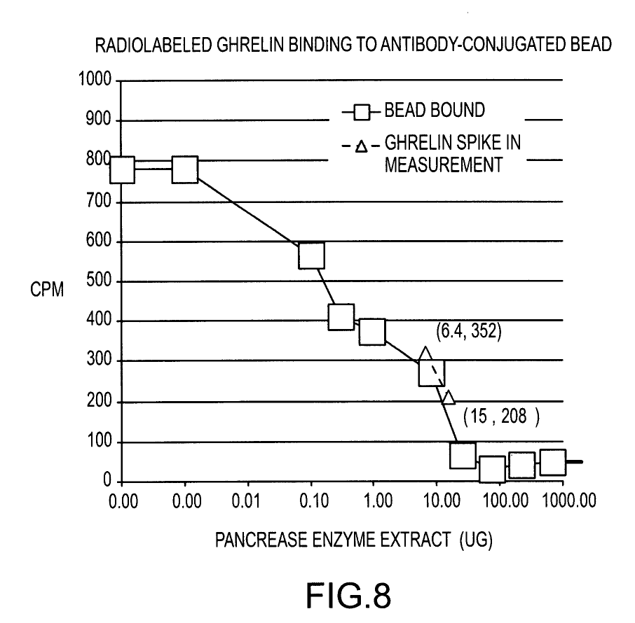

Now referring primarily FIG. 8, which shows that embodiments of the inventive immunoassay particle (2) do not bind non-target particles (8) in the immunoassay sample (6) such as degradation fragments of labeled particle (9). I¹²⁵-ghrelin peptide was incubated with a bovine pancrease enzyme and the measure of dissociable capture of the treated I¹²⁵-ghrelin to the anti-ghrelin antibody attached to the solid phase substrate (3) was made in accordance with the inventive method. As can be understood from FIG. 8, the binding of I¹²⁵-ghrelin to anti-ghrelin antibody correspondingly decreases in relation to the concentration of bovine pancrease enzyme introduced into matched I¹²⁵-ghrelin samples and incubated for 1 hour incubation.

Because the inventive immunoassay particle (2) does not non-specifically bind fragments of degraded labeled particles (9) but continues to specifically bind fragments with intact epitopes, the inventive immunoassay particle (2) can be used to screen protein digests to identify the epitopes which bind particular antibodies. Generally speaking, the invention allows for the identification of antibodies which bind specific protein fragments or the identification of specific protein fragments which bind specific antibodies utilized as the target particle capture moiety (4) of the immunoassay particle (2). The protein fragments can be generated by protease cleavage of a peptide or protein using various proteolytic enzymes. Typically, the smallest synthetic peptide that will consistently elicit the production of antibodies that bind the original peptide or protein are about 6 residues in length. Certain protease cleavage sites can be predicted using a computer program, such as Peptidecutter, to analyze the peptide or protein to be treated with one or more proteases.

For example, ghrelin peptide in the naturally occurring form has 28 residues. Using a computer program such as Peptidecutter for analysis of cleavage, trypsin can cleave at positions 10,14,15,18, 23 and 27. Thermolysin can cleave at positions 2, 3, 10, 21, and 23. The anti-ghrelin antibody utilized to produce the results summarized by FIG. 8 binds the C-terminal region of the ghrelin peptide comprising residues 15 to 24. As such, the loss of radioactive-ghrelin bound to the anti-ghrelin antibody represents a degradation in the region of the peptide between residues 15 to 24 resulting from cleavage by the enzyme trypsin.

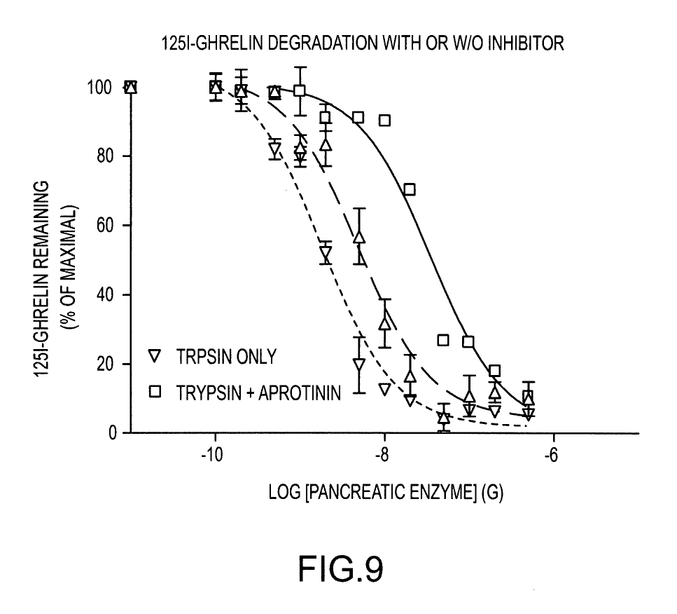

Now referring primarily to Example 11 and FIG. 9, embodiments of the inventive immunoassay system (1) allows for the measurement of inhibition of peptide degradation by enzyme inhibition. As shown by FIG. 9, the ghrelin peptide can be incubated with pancreatic enzyme extract only at a plurality of concentrations in the absence of protease inhibitor (trypsin only utilized in Example 11), or in the presence of heat inactivated protease inhibitor (heat deactivated aprotinin utilized in Example 11) or in the presence of the protease inhibitor (aprotinin utilized in Example 11). As can be understood from the data presented in FIG. 9, protease inhibition of trypsin can be assessed by an increased measure of binding by the ghrelin peptide to the anti-ghrelin antibody of the immunoassay particle.

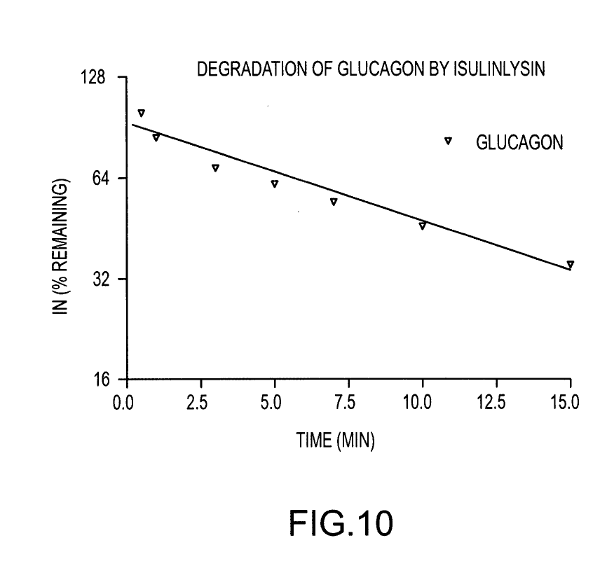

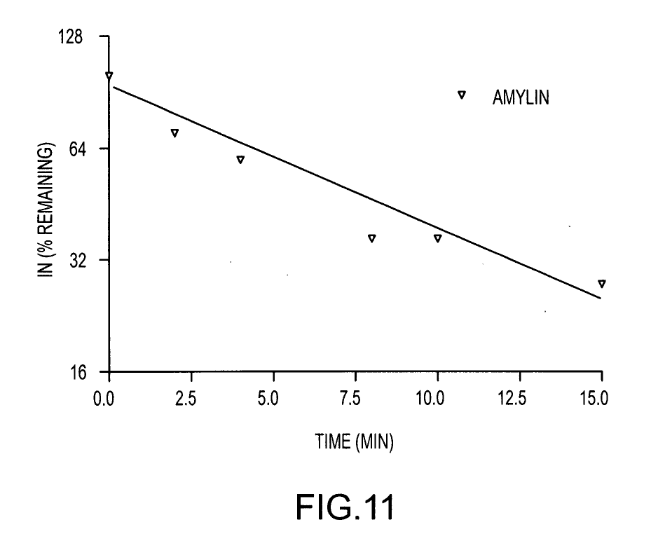

Now referring primarily to FIGS. 10, 11 and 12 and Example 12, an embodiment of the inventive immunoassay system (1) allows for the coincident measurement of the degradation of a plurality of target particles (7) contained in the same immunoassay sample (6). First referring to FIG. 10, which shows the results of utilizing the inventive immunoassay method to determine decreased concentration of glucagon in an immunoassay sample (6) upon degradation with insulysin (“IDE”). As can be understood, the inventive immunoassay shows that the concentration of glucgon decreases as the duration of time the glucagon combined with the insulysin increases. Similarly, now referring to FIG. 11, the inventive immunoassay method can be used to determine the decreased concentration of amylin in an immunoassay sample (6) upon degradation by IDE.

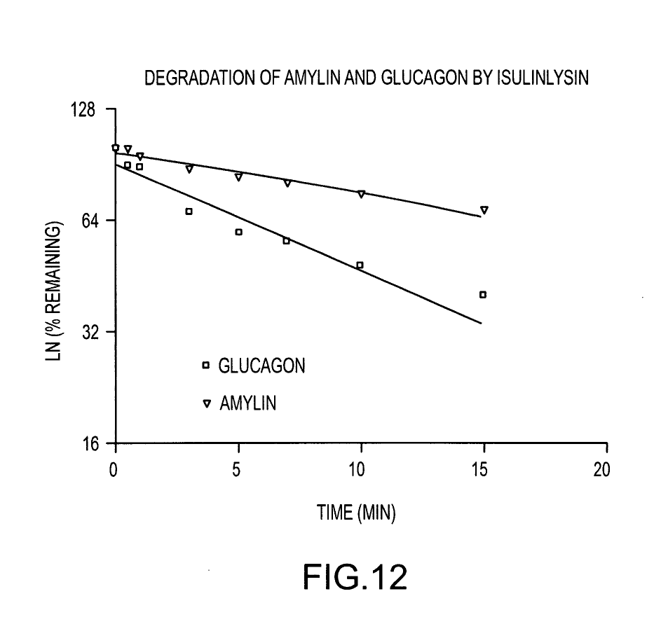

As shown in FIG. 12 and further described by Example 12, the coincident determination of the decrease in glucagon and amylin in a single immunoassay sample (6)(as evidenced by the reduced binding of radiolabeled glucagon and radiolabeled amylin to a corresponding immunoassay particles (2) upon digestion with insulin-degrading enzyme (“IDE”) can be achieved using the Multiplex Assay embodiment of the invention. The prior conventional immunoassays, whether RIA, ELISA, or EIA only allows the assay of a single protein or peptide degradation in a immunoassay sample (6).

### EXAMPLE 1

### Conventional RIA Method

Conventional RIA methods can include the following steps:

### 1. Combine 100 μL of a conventional immunoassay sample or standard containing target particles and 100 μL rabbit antibody solution into an assay tube.

### 2. Mix the resulting combination and incubate overnight (16-24 hrs) at 4° C.

3. Add 125I-labeled particle (typically an amount of 125I-labeled protein or peptide generating about 15,000 counts per minute by gamma counter) prepared by reaction of Na¹²⁵I with the target particle (about 1 mCi per 100 μg of purified peptide or protein) and purification of the 125I-labeled particle by application to column chromatography gel (Bio-Gel P30 Bio-Rad #150-4154). Mix and incubate overnight (16-24 hrs.) at 4° C.

### 4. Add 100 μL of goat anti-rabbit IgG serum solution and normal rabbit serum.

### 5. Mix and incubate at room temperature for 90 to 120 minutes to precipitate binding complex aggregates.

### 6. Add radioimmunoassay buffer (“RIA buffer”)(typically 150 mM NaCl, 100 mM sodium phosphate, 1% BSA and 1% Triton X-100). Mix and centrifuge for 20 minutes at 1,700×g.

### 7. Aspirate off the supernatant (except for the tubes used to determine total counts).

### 8. Measure counts per minute for each immunoassay sample.

### 9. Calculate B/B0 for each immunoassay sample.

### 10. Determine concentration of target particle in original immunoassay sample.

Total assay time between about 36 hours to about 48 hours.

Now referring to FIG. 2 which provides an example of the results obtained from conventional RIA. The plasma concentration interpolated from standard curves and indicate about 18.8 pg in 75 μL.

### EXAMPLE 2A

### Magnetic Solid Phase Substrate Linked Antibody

A particular embodiment of the immunoassay particle (2) can be prepared as follows:

BIOMAG beads responsive to a magnetic field having a mean diameter of about 1.5 μm were obtained from Polysciences, Inc., 400 Valley Road, Warrington, Pa. 18976. The BIOMAG beads were activated by glutaraldehyde in 0.01 M pyridine immediately prior to conjugation of the antibody to the BIOMAG beads. Incubation of the BIOMAG beads in the presence of an antibody in 0.01 M pyridine overnight at 21° C. (1 to 500 μg antibody per mL supernatant depending upon the antibody conjugated and specifically with respect to anti-ghrelin polyclonal antibody about 500 μg/mL). The antibody-conjugated BIOMAG beads were separated from the supernatant by application of magnetic force. The separated BIOMAG bead conjugate was then incubated in 1 M glycine (pH 8.0) and 0.1% w/v BSA for 1 hr. on an orbital shaker. The antibody-conjugated bead was then washed five times with a wash buffer (0.01M Tris. 0.15M NaCl, 0.1% w/v BSA and 0.001 M EDTA) and then suspended in a solution containing 0.15M NaCl, 0.01M Tris, 0.1% BSA and 0.05% NaN3, pH 7.4, for storage.

The use of the BIOMAG bead as described is not intended to be limiting with respect to the numerous and varied solid phase substrates which can be utilized in the inventive immunoassay system (1) but rather provides an example sufficient for the person of ordinary skill in the art to make and use numerous embodiments of the inventive immunoassay system (1).

### EXAMPLE 2B

### Non-Magnetic Solid Phase Substrate Linked Antibody

A particular embodiment of the immunoassay particle (2) can be prepared as follows:

Amino polystyrene beads having a mean diameter of about 11.2 μm were obtained from Spherotech, Inc., 1840 Industrial Dr. Suite 270, Libertyville, Ill. 60048. The amino polystyrene beads were activated by glutaraldehyde in 0.01 M pyridine immediately prior to conjugation of the antibody to the non-magnetic bead. Incubation of the non-magnetic beads in the presence of an antibody (such as 500 μg/mL anti-ghrelin polyclonal antibody) in 0.01 M pH 7.0 phosphate buffer overnight at 21° C. The antibody-conjugated non-magnetic beads was separated from the supernatant by centrifugation at 3000× g. The separated non-magnetic bead conjugate was then incubated in 200 mM ethanolamine (pH 7.0) and 0.1% w/v BSA for 1 hr. on an orbital shaker. The antibody-conjugated bead was then washed five times with a wash buffer (0.01M Tris. 0.15M NaCl, 0.1% w/v BSA and 0.001 M EDTA) and then suspended in a solution containing 0.15M NaCl, 0.01M Tris, 0.1% BSA and 0.05% NaN3, pH 7.4, for storage.

The use of the amino polystyrene bead as described is not intended to be limiting with respect to the numerous and varied solid phase substrates which can be utilized in the inventive immunoassay system (1) but rather provides an example sufficient for the person of ordinary skill in the art to make and use numerous embodiments of the inventive immunoassay system (1).

### EXAMPLE 3

### Radiolabeled Ghrelin Binding by Solid Phase Substrate Linked Antibody

A particular embodiment of the immunoassay method for dissociable capture of radiolabeled ghrelin peptide by solid phase linked antibody can be performed as follows:

1. Combine 100 ul of I¹²⁵-ghrelin (specific activity 3.15 p mole/ml) prepared in accordance with Example 1.3 (Example 1 step 3) to each of eight aliquots of 50 μL antibody-conjugated beads and to each of two aliquots of 50 μL glycine-conjugated beads prepared in accordance with Example 2B suspended in stock solution (0.01 M Tris (pH 7.4), 0.1% NaN₃, 0.1% w/v bovine serum albumin (BSA), 0.15M NaCl, and 0.001M EDTA.

### 2. Incubate antibody-conjugated beads at room temp (21° C.) for 0, 5, 10, 15, 30, 60, 120, and 249 min.

3. To each incubated mixture of antibody-conjugated beads and I¹²⁵-ghrelin add 1.5 ml RIA buffer (150 mM NaCl, 100 mM sodium phosphate, 1% BSA and 1% Triton X-100), mix, and centrifuge for 5 minutes at 17,000× g. Aspirate off the supernatant.
4. Wash once with 1 ml RIA buffer. Aspirate off the supernatant

### 5. Measure and record counts per minute for each assay tube.

Now referring primarily to FIG. 3, the time course of I¹²⁵-ghrelin binding to anti-ghrelin antibody-conjugated bead (▴) and non-specific binding control of glycine-conjugated bead () shows a two hour saturation binding for antibody-conjugated beads. The results shown are the means of duplicates. The counts from gamma counter represent the specific binding by anti-ghrelin antibody. The binding curve was generated using a non-linear least square fitting program in GraphPad Prism (version 2.01).

### EXAMPLE 4

### 150 Microliter Small Volume Competitive Radiolabeled Ghrelin Binding by Solid Phase Substrate Linked Antibody

A particular embodiment of the immunoassay method for competitive dissociable capture of radiolabeled ghrelin peptide by solid phase linked antibody to determine ghrelin peptide concentration in a 150 μl small volume immunoassay sample can be performed as follows:

### 1. Combine 25 μl of antibody conjugated beads in stock solution prepared in accordance with Example 2B to 50 μl human plasma or 50 μl standard solution.

### 2. Mix the resulting combination and incubate 6 hrs at 4° C.

### 3. Add 25 μl of I¹²⁵-ghrelin prepared in accordance with Example 1.3 and 50 μl RIA buffer.

### 4. Incubate 16-18 hrs. at 4° C.

5. Add 1.5 ml RIA buffer to incubated mixture and centrifuge for 5 minutes at 17,000× g. Aspirate off the supernatant (except total counts tubes).
6. Wash once with 1 ml RIA buffer. Aspirate off the supernatant (except total counts tubes).

### 7. Measure and record counts per minute for each assay tube.

### Total assay time about 24 hours for this embodiment of the inventive immunoassay method.

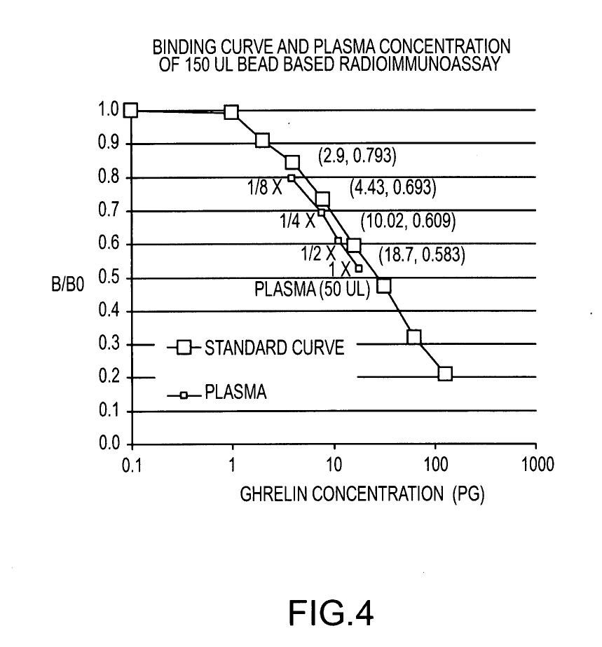

Now referring primarily to FIG. 4 which shows the results of bead-based RIA which utilizes a total volume of about 150 μl: 25 μl of beads to combine with 50 μl of test sample, to which add 25 μl of I¹²⁵-ghrelin, 50 μl of assay buffer for a total 150 μl immunoassay volume. The plasma concentration was interpolated from standard curves and indicate about 18.7 pg in 150 ul solution. The amounts of isotope labeled-ligands applied (as indicated in total counts tubes) was twice that of the conventional RIA described in Example 1. The results of this embodiment of the inventive bead-based RIA are comparable to the conventional RIA method of Example 1 having an IC50 of about 400 pg/ml and maximal count at 23000 cpm. The total assay time was about 24 hours for this embodiment of the inventive immunoassay method. While this particular embodiment of the inventive immunoassay method utilizes a total volume of about 150 μl and a total assay time of about 24 hours (“one day”), this example is not intended to limit the inventive immunoassay system to a particular volume or total volume but rather is intended to be illustrative of the reduce volumes and reduced total assay times achievable with the inventive immunoassay system (1).

### EXAMPLE 5

### 60 Microliter Small Volume Competitive Radiolabeled Ghrelin Binding by Solid Phase Substrate Linked Antibody

A particular embodiment of the immunoassay method for competitive dissociable capture of radiolabeled ghrelin peptide by solid phase linked antibody to determine ghrelin peptide concentration in a 60 μl small volume immunoassay sample can be performed as follows:

### 1. Combine 25 μl of antibody immobilized beads in stock solution prepared in accordance with Example 2B to about 25 μl human plasma or 10 μl-25 μl standard solution.

### 2. Mix resulting combination and incubate 6 hours at room temperature.

3. Add 10 μl 125I-peptide. Mix and incubate overnight (16-18 hrs.) at 4° C.
4. Add RIA buffer. Mix and centrifuge for 5 minutes at 1,700 g. Aspirate off the supernatant (except total count tubes).
5. Wash once with 1 ml RIA buffer. Aspirate off the supernatant (except total count tubes).

### 6. Measure and record counts per minute for each assay tube.

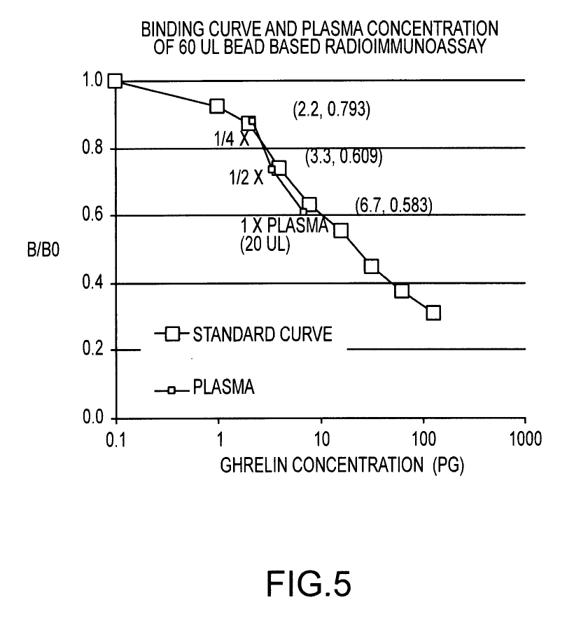

FIG. 5 shows the result of the inventive bead-based RIA which utilizes a total volume of about 60 μL: 25 μL of beads in stock solution combined with about 25 μL of test sample and 10 μL of I¹²⁵-ghrelin to generate a 60 μL total immunoassay sample volume. The plasma concentration was interpolated from standard curves and indicate about 18.7 pg in 150 μl solution. The amounts of isotope labeled-ligands applied (as indicated in total counts tubes) was twice that of the conventional RIA as described in Example 1. The results of this embodiment of the inventive bead-based RIA are comparable to the conventional RIA exemplified in EXAMPLE 1 having an IC50 around 400 pg/ml and maximal count at 23000 cpm. While this particular embodiment of the inventive immunoassay method utilizes a total volume of about 60 μl and a total assay time of about 24 hours, this example is not intended to limit the inventive immunoassay system to a particular volume or total but rather is intended to be illustrative of the reduce volumes and reduced total assay times achievable with the inventive immunoassay system (1).

### EXAMPLE 6

### Comparison of One Day Small Volume Competitive Radiolabeled Nesfatin Binding by Solid Phase Substrate Linked Antibody to Conventional Radioimmunoassay.

A particular embodiment of a one day small volume inventive immunoassay method for competitive dissociable capture of radiolabeled nesfatin peptide by solid phase linked antibody to determine nesfatin peptide concentration in rat plasma can achieve results comparable to conventional RIA methods.

An aliquot of 10 μL of magnetic beads prepared in accordance with Example 2A suspended in stock solution was combined with 10 μL of RIA buffer in 5 mL assay tubes. The resulting 20 μL mixture was combined with 20 μL of 100 μL or 200 μL, or 400 μL of rat plasma or with nesfatin standard and incubated at room temperature on an orbital shaker 250 rpm for 2 hr. The assay tubes were then placed in a magnetic field of sufficient strength to generate a pellet of the magnetic beads. The volume of supernatant was adjusted by aspiration to 40 μl and brought to 60 μl by addition of 20 μL of I¹²⁵-labeled nesfatin. After the incubation with I¹²⁵-nesfatin 2 hr, the dissociable capture of nesfatin were terminated by addition of 1.5 ml of RIA buffer. The magnetic beads were centrifuged for 20 min. at 1,700× g. The resulting pellet of magnetic beads was re-suspended with 1.5 ml of RIA buffer and centrifuged for 20 min. at 1,700×g. The supernatant was removed and the resulting pellets of magnetic beads were place in a Gamma counter to obtain counts per minute. The conventional RIA method of Example 1 was performed with 200 μL and 400 μL aliquots of rat plasma for comparison.

Using the conventional RIA of Example 1, the plasma concentration of nesfatin was interpolated from standard curves as about 1624±32 pg/mL. The immunoassay method utilizing the small volume immunoassay samples to determine the concentration of nesfatin in rat plasma yielded results of 1879±81, 1406±325, and 1937±66 pg/ml with respect to the corresponding 100 μL, 200 μL, and 400 μL immunoassay samples. The results of this embodiment of the inventive bead-based immunoassay are comparable to those obtained by the conventional RIA method of Example 1. However, there are substantial advantages in using the inventive immunoassay system because the assay time can be reduced from about three days to about one day.

### EXAMPLE 7

### Enrichment of Ghrelin Peptide in Human Saliva With Quantitative Measurement

A particular embodiment of a one day small volume inventive immunoassay method for competitive dissociable capture of radiolabeled ghrelin peptide by solid phase linked antibody was utilized to determine effects of immunoassay sample dilution on determination of ghrelin peptide concentration in human saliva.

Human saliva was collected and heated to 100° C. for 5 min and subsequently centrifuged for 10 min. at 15000 rpm. 0.5 mL of saliva sample or physiologic saline control were incubated with 20 μL of magnetic beads conjugated with anti-ghrelin antibody as described in Example 2A on an orbital shaker for 3 hr at room temperature. After incubation, the magnetic beads were separated by application of a magnetic field of sufficient strength to aggregate the magnetic beads in the saliva sample. An aliquot of 480 μL of the saliva sample was removed resulting in a 40 μL volume. An aliquot of 20 μL of 125I labeled ghrelin was added and incubated at 4° C. for 12 hr. The dissociable capture of 125I labeled ghrelin was terminated by addition of 1.5 ml of RIA buffer. The magnetic beads were centrifuged for 20 min. at 1,700×g. The resulting pellet of magnetic beads was resuspended with 1.5 ml of RIA buffer and centrifuged for 20 min. at 1,700×g. The supernatant was removed and the resulting pellets of magnetic beads were place in a Gamma counter to obtain counts per minute. The determined ghrelin concentration in human saliva samples by interpolation to a standard curve was 81.5 pg/ml ghrelin.

To compare the effect of different incubation volumes in determining ghrelin concentration in the inventive immunoassay system, samples of human saliva of 20 μL, 40 μL, and “20 μL diluted to 0.5 mL” were each assayed. Each human saliva sample was assayed as above described in this Example 7. The results in Table 3 indicate that a broad range of the dilution values can be utilized in the inventive immunoassay method in determining concentration of target particles in samples using the inventive immunoassay methods.

### EXAMPLE 8

### Enrichment of Ghrelin Peptide in Human Plasma With Quantitative Measurement

A particular embodiment of a small volume inventive immunoassay method for competitive dissociable capture of radiolabeled ghrelin peptide by solid phase linked antibody was utilized to determine effects of immunoassay sample dilution on determination of ghrelin peptide concentration in human plasma.

Human plasma was collected and a 20 μL aliquot, a 20 μL aliquot (brought to 60 μL with RIA buffer), and a 20 μL aliquot (brought to 500 μL with RIA buffer) were transferred to immunoassay tubes. A volume of 20 μL of immunoassay beads prepared in accordance with Example 2B suspended in stock solution was added to each assay tube. Standard samples and a 0.5 mL of peptide free plasma was spiked with four picograms ghrelin as a positive control for recovery rate calculation were prepared according to the same protocol. The resulting mixture in each assay tube was incubated on an orbital shaker for 3 hr at 300 rpm at room temperature. The incubated mixtures were centrifuged for 3 min at 15000 rpm for a brief duration to establish a pellet of immunoassay beads. From the 20 μL aliquot of human plasma brought to 60 μL with RIA buffer, 40 μL of the supernatant was removed, and from the 20 μL aliquot of human plasma brought to 500 μL with RIA buffer 460 μL was removed. Then 20 μL of I¹²⁵-labeled ghrelin was added to bring the volume of each immunoassay sample to a total volume of 60 μL accordingly. The mixture in each immunoassay tube was incubated for a duration of 18 hrs on an orbital shaker at 250 rpm at 4° C. The incubated mixtures were centrifuged at 15000 rpm for 15 min to pellet the immunoassay beads and the supernatant was removed. The pellet was washed with 1.5 ml of RIA buffer and centrifuged at 15000 rpm for 15 min pellet the immunoassay beads. The wash solution was aspirated and the CPM was counted by gamma counter.

The results are set out in Table 4, a volume of 20 μL, 60 μL, and 500 μL and the final assay results of plasma ghrelin were 126, 118, and 135 pg/mL from 20 μL, 60 μL, and 500 μL, respectively which indicate that consistent results can be generated by the inventive immunoassay method regardless of variation in sample volume. Additionally, very dilute samples can be enriched by use of embodiments of the inventive immunoassay method to achieve results consistent with the assay in small volume of sample.

### EXAMPLE 9

### Two Day Small Volume Competitive Radiolabeled Binding by Solid Phase Substrate Linked Antibody To Assess Obestatin Peptide Concentration in Human Plasma

A particular embodiment of a two day small volume inventive immunoassay method for competitive dissociable capture of radiolabeled obestatin peptide by solid phase linked antibody was utilized to determine the range of linear detection.

Aliquots of human plasma of 5 μL (diluted to 20 μL with RIA buffer 1:4), 10 μL (diluted to 20 μL with RIA buffer 1:2), 20 μL or 100 μL were combined with 20 μL of antibody conjugated beads suspended in stock solution prepared in accordance with Example 2B. The resulting mixtures were incubated at 4° C. on a orbital shaker 250 rpm for 12-16 hrs and centrifuged at 15000 rpm 15 min. to pellet the antibody conjugated beads. A 20 μL aliquot of the supernatant was removed from each assay tube and replaced with a 20 μL aliquot of radio-labeled obestain. The resulting mixtures were incubated at 4° C. on an orbital shaker 250 rpm for 12-16 hrs. and then centrifuged to pellet the antibody conjugated beads. The supernatant was removed and the pellets of antibody conjugated beads were washed with 1.5 mL of wash solution followed by centrifugation at 15000 rpm 15 min. to pellet the antibody conjugated beads. The wash solution was removed and the pelleted antibody conjugated beads and the CPM counted by gamma counter. The counts were interpolated by comparison to a standard curve and the results were summarized in FIG. 7. The linear detection range of the inventive immunoassay method included the 10 μL, 20 μL, and 100 μL samples. The 100 ul sample being closest to the IC50. Understandably, where a sample is expected to have a level of target particle close to the detection limit of the inventive immunoassay it may be appropriate to obtain a sample volume, if possible.

### EXAMPLE 10

### Multiplex Detection of Orexin, Ghrelin, and Obestatin in Human Plasma

A particular embodiment of a small volume inventive immunoassay method for competitive dissociable capture of radiolabeled ghrelin peptide by solid phase linked antibody was utilized to determine the concentration of three target particles in a single sample.

Aliquots of 200 uL or 60 uL of human plasma or physiologic saline control were incubated with 20 μL antibody-conjugated magnetic bead prepared in accordance with Example 2A in stock solution for dissociable capture of orexin A and antibody-conjugated non-magnetic beads prepared in accordance with Example 2B in stock solution for dissociable capture of ghrelin on an orbital shaker for 3 hr in room temperature. The antibody-conjugated magnetic beads were separated from the supernatant by application of magnetic field and introduced into a “first target particle assay” to determine concentration of orexin A. The remaining non-magnetic beads were separated from the supernatant by centrifugation at 2000×g for 10 min and introduced into a “second target particle assay”. The supernatant was saved and a portion transferred to each of the first target particle assay, the second target particle assay, and into the conventional RIA method of Example 1 as a “third target particle assay” as further described below.

### First Target Particle Assay.

To each sample of magnetic beads separated as above described, add 20 μL supernatant correspondingly collected from that sample. Subsequently, add 20 μL 125-I labeled orexin peptide prepared in accordance with Example 1.3 to each sample of magnetic beads to generate a total volume of about 60 μL. Incubate the resulting mixture for 8-12 hr. Pellet the magnetic beads by application of magnetic field or by centrifugation at 15000 rpm 15 min. and remove the supernatant by aspiration. After removal of the supernatant, the magnetic bead pellets are washed with 1.5 ml of RIA buffer and centrifuged at 15000 rpm 15 min pellet the magnetic beads and the RIA buffer removed. The counts per minute for each magnetic bead pellet was obtained by gamma counter.

### Second Target Particle Assay.

To each sample of non-magnetic beads separated as above described, add 20 μL supernatant correspondingly collected from that sample. Subsequently, add 20 μL 125-I labeled ghrelin peptide prepared in accordance with Example 1.3 to each sample of non-magnetic beads to generate a total volume of about 60 μL. Incubate the resulting mixture for 8-12 hr. Pellet the non-magnetic beads by centrifugation at 15000 rpm 15 min. and remove the supernatant by aspiration. After removal of the supernatant, the non-magnetic bead pellets are washed with 1.5 ml of RIA buffer and centrifuged at 15000 rpm 15 min pellet the magnetic beads and the RIA buffer removed. The counts per minute for each non-magnetic bead pellet was obtained by gamma counter.

### Third Target Particle Assay.

For each sample transfer 100 μL of the supernatant to an assay tube. Add 100 μL rabbit antibody solution and incubate 8-12 hrs at 4° C. Add 100 μL 125-I labeled third target particle to each assay tubes and incubate 8-12 hrs at 4° C. Then add 100 μL goat anti-rabbit antibody and 100 μL of normal rabbit serum to each assay tube and incubate at room temperature for 2 hr. Add 0.5 ml RIA buffer to each sample assay tube. Centrifuge for 20 min and aspirate off the supernatant from sample assay tubes and standard tubes but not total counts tubes. Determine CPM by gamma counter and interpolate counts to the standard curve to determine concentration of the third target particle in the original sample.

### EXAMPLE 11

### Measuring Inhibition of Peptide Degradation

Three different digestion conditions were established to measure the inhibition of degradation of the ghrelin peptide by the inventive immunoassay system. The first digestion condition established a plurality of ghrelin samples digested with increasing concentration of trypsin enzyme without any inhibitor for 1 hr at 37° C.

The second condition established a plurality of ghrelin samples digested with increasing concentration of trypsin enzyme in the presence of peptide free heat inactivated human serum (“HS”) purchased from Scantianbodies Laboratory Inc. in Santee, Calif. The HS prior mixed with active charcoal (7% w/w) for 16 hours in room temperature and then centrifuged at 16000× g followed by filtration (0.22 um filter) to remove particulates and peptides. 20 μL of peptide free HS was then added to each digestion sample having different concentration of trypsin and then incubated for 1 hr at 37° C.

The third condition established a plurality of ghrelin samples digested with increasing concentration of trypsin enzyme in the presence of the protease inhibitor Aprotinin (0.3 TIU/ml), a bovine lung kallikrein inactivator purchased from Calbiochem in San Diego, Calif.

As to each digestion condition, the initial time was recorded and a 1 hr digestion period was allowed to elapse before adding of 10 μl of PMSF and 250 μl of 0.02M ammonium chloride (pH 5.0) to stop the digestion reaction. Then, each ghrelin sample digest was heated for 15 min at 85° C. and then frozen immediately on dry ice. The inventive RIA was carried by combining for each of the plurality of ghrelin digested samples 50 μL immunoassay bead presenting anti-ghrelin antibody as the capture moiety with 150 μL of radio-labeled ghrelin mixture for a total volume of 300 μL for each sample. After incubation of 1 hour, those tubes were centrifuged at 1700× g for 10 min to generate pellets. The pellets were washed with 1.5 ml of radioimmunoassay buffer once and the counts taken by gamma counter.

### In Summary, the Assay Procedure For Measuring Ghrelin Peptide Degradation as Following:

### 1. Prepare by serial dilution each desired concentration of an enzyme in NaHCO₃ solution (pH8.0). Aliquot in duplicate 150 μL of each concentration of the enzyme solution to 1.5 mL eppendorf tubes.

### 2. To each 150 μL volume of each concentration of such enzyme solution add I¹²⁵-labeled peptide to incubate at 37° C. for 30 min.

### 3. Add 300 μl of 10 mM ammonium chloride (pH 4.0) and heat at 85° C. for 15 min to stop the reaction.

### 4. Combine 50 μl immunoassay bead, 120 μL RIA buffer, 50 μl aprotinin to 80 μl of digested I¹²⁵-labeled peptide solution and incubate for 30 min with agitation.

### 5. Centrifuge 1700×g for 5 min to pellet the beads and discard the supernatant.

### 6. Add 1 ml of RIA buffer and centrifuge at 1700×g for 5 min.

### 7. Suction off the supernatant and use a gamma-counter to count the CPM of the pellet

Specifically, with regard to the results shown in FIG. 6, ghrelin degradation was achieved by utilizing increasing concentrations of purified bovine pancreas enzyme. The effect of the enzyme on ghrelin as measured by the reduction of bound radiolabel ghrelin to anti-ghrelin antibody.

### EXAMPLE 12

### Multiplex Measurement of Peptide Degradation

Prior to performing the multiplex assay single peptide degradation plots were prepared for each of glucagon shown in FIG. 10 and for amylin shown in FIG. 11.

### Single Peptide Assay Procedure.

### 1. Prepare separately solutions of 200 pg/mL of human glucagon (adjust with I¹²⁵-glucagon) and 40 pg/mL of human amylin (adjust with I¹²⁵-amylin) each in a total volume of 2 mL.

### 2. Prepare a digestion solution of insulysin (IDE) 6.2 μg in 3.1 mL of 0.15M NaCl, 25 mM Tris, pH7.5. Aliquote 0.4 mL to a tube for total of 12 tubes of digestion solution.

### 3. Add to the digestion solution with above 0.4 mL of human glucagon or amylin solution.

### 4. Incubate at 37° C. for a time interval at 15, 10, 7, 5, 3, 1 min and a tube without the incubation.

### 5. At the indicated time, add 40 μL protease inhibitor (bacitracin 2 mg/ml final concentration) and 300 μL of 10 mM ammonium chloride to stop the reaction.

### 6. Transfer 50 μL antibody conjugated bead prepared in accordance with Example 2B, 120 μL RIA buffer, 50 μl aprotinin and 80 μL of digested I¹²⁵-labeled peptide solution to 1 mL conical tube and incubate with agitation for 30 min.

### 7. Spin 5 min at 16000×g to pellet the beads and discard supernatant.

### 8. Add 1 mL of RIA buffer and centrifuge at 1700×g for 5 min.

### 9. Remove the supernatant and count the CPM of the pellet by gamma counter.

As shown by FIG. 10, the results of the means of duplicates show degradation of human glucagon assessed by the inventive immunoassay system and exhibits pseudo-first order degradation rate constants and half life estimated by using a linear regressiom curves of pseudo-first order kinetic plot for degradation generated by using GraphPad Prism (version 2.01) by with a pseudo-first order model. T_{1/2}=ln2/kₐₚₚ.

As shown by FIG. 11, the results of the means of duplicates show degradation of human amylin assessed by the inventive immunoassay system and exhibits pseudo-first order degradation rate constants and half life estimated by using a linear regressiom curves of pseudo-first order kinetic plot for degradation generated by using GraphPad Prism (version 2.01) by with a pseudo-first order model. T_{1/2}=ln2/kₐₚₚ.

### Multiplex Peptide Assay Procedure.

### 1. Prepare a 2 μg/ml concentration of insulysin (IDE) as the digestion solution.

2. n each tube, 200 μL of above digestion solution add with I¹²⁵-labeled peptide to incubate at 37° C. at the indicated time of 15, 10, 5, 3, 1 min to stop reaction and a tube of no incubation as the time zero.

### 3. Add 40 μL bacitracin (for a final concentration of 2 mg/ml) and 300 μL of 10 mM ammonium chloride to stop the reaction.

### 4. Pipette 50 μL antibody conjugated bead in accordance with Example 2B, 120 μL RIA buffer, 50 μL bacitracin (for a final concentration of 1 mg/ml) and 80 μL of digested I¹²⁵-labeled peptide solution to incubate on orbital shaker for 30 min.

### 5. Spin 5 min at 1700×g to pellet the beads and discard supernatant.

### 6. Add 1 ml of RIA buffer and centrifuge 5 min at 1700×g.

### 7. Remove the supernatant and assess CPM of the pellet using a gamma-counter.

FIG. 12, provides a graph of the results of utilizing multiplex detection of the degradation of glucagon and amylin coincident in time using 2 μg/ml of insulysin (IDE) for Glucagon (□) and amylin (▾). Each immunoassay sample contained 300 μL of 200 pg/mL glucagon and 40 pg/ml amylin in the beginning of the experiment. Data shown are averages of duplicates. The rate constants of degradation were estimated by using a linear Liner regression curves of pseudo-first order kinetic plot for degradation are generated by using GraphPad Prism (version 2.01) by with a pseudo-first order model. T_{1/2}=ln 2/kₐₚₚ

As can be easily understood from the foregoing, the basic concepts of the present invention may be embodied in a variety of ways. The invention involves numerous and varied embodiments of an inventive bead-based radioimmunoassay and methods of using such embodiments of the inventive bead-based radioimmunoassy.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “assay” should be understood to encompass disclosure of the act of “assaying”—whether explicitly discussed or not—and, conversely, were there effectively disclosure of the act of “assaying”, such a disclosure should be understood to encompass disclosure of an “assay” and even a “means for assaying.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the bead-based radioimmunoassy devices or systems herein disclosed and described, ii) the related methods disclosed and described, iii) similar, equivalent, and even implicit variations of each of these devices and methods including but not limited to ELISA and EIA, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

We claim:

1. A method of radioimmunoassay, comprising the steps of:
a) providing an amount of immunoassay particle having a solid phase substrate linked to an amount target particle capture moiety;
b) providing an amount of immunoassay sample which provides an amount of liquid which entrains an amount of target particle capable of dissociable capture by said target particle moiety linked to said solid phase substrate of said immunoassay particle;
c) providing an amount of labeled particle capable of disossociable capture by said target particle moiety linked to said solid phase substrate of said immunoassay particle;
d) combining said amount of labeled particle with said amount of immunoassay particle and said amount of immunoassay sample and to generate a volume of not greater than 150 microliters;
e) incubating the combination of said amount of immunoassay particle and said amount of immunoassay sample and said amount of labeled particle for an interval of time;
f) establishing a constant rate of disossociable capture of each of said target particle and said labeled particle with said amount of target particle capture moiety during said interval of time;
g) separating said amount immunoassay sample and said amount of labeled particle from said amount of immunoassay particle;
h) determining a fractional part of said amount of labeled particle captured by said amount of particle capture moiety; and
i) assessing said amount of target particle in said amount of immunoassay sample.

2. The method of radioimmunoassay as described by claim 1, wherein said step of combining said amount of labeled particle with said amount of immunoassay particle and said amount of immunoassay sample and to generate a volume of not greater than 150 microliters comprises the step of combining said amount of immunoassay particle and said amount of immunoassay sample and said amount of labeled particle to generate a volume selected from the group consisting of: a volume of between about 25 microliters and about 35 microliters, a volume of between about 30 microliters and about 40 microliters, a volume of between about 35 microliters and about 45 microliters, a volume of between about 40 microliters and about 50 microliters, a volume of between about 45 microliters and about 55 microliters, a volume of between about 50 microliters and about 60 microliters, a volume of between about 55 microliters and about 65 microliters, a volume of between about 60 microliters and about 70 microliters, a volume of between about 65 microliters and about 75 microliters, a volume of between about 70 microliters and about 80 microliters, a volume of about 75 microliters and about 85 microliters, a volume of between about 80 microliters and about 90 microliters, a volume of between about 85 microliters and about 95 microliters, a volume of between about 90 microliters and about 105 microliters, a volume of between about 100 microliters and about 110 microliters, a volume of between about 105 microliters and about 115 microliters, a volume of between about 110 microliters and about 120 microliters, a volume of between about 115 microliters and about 125 microliters, a volume of between about 120 microliters and about 130 microliters, a volume of between about 125 microliters and about 135 microliters, a volume of between about 130 microliters and about 140 microliters, a volume of between about 135 microliters and about 145 microliters, a volume of between about 140 microliters and about 150 microliters, and a volume of between about 145 microliters and about 155 microliters,

3. The method of radioimmunoassay as described by claim 2, wherein said step of providing an amount of immunoassay particle having a solid phase substrate linked to an amount target particle capture moiety comprises the step of providing an amount of immunoassay particle having a solid phase substrate linked to an amount target particle capture moiety, wherein said immunoassay particle has a mean diameter of between about 0.5 micrometer and about 15 micrometers.

4. The method of radioimmunoassay as described by claim 3, wherein said step of providing an amount of immunoassay particle having a solid phase substrate linked to an amount target particle capture moiety, wherein said immunoassay particle has a mean diameter of between about 0.5 micrometer and about 15 micrometers comprises the step of providing an amount of magnetic immunoassay particle having a solid phase substrate linked to an amount target particle capture moiety, wherein said immunoassay particle has a mean diameter of between about 0.5 micrometer and about 15 micrometers.

5. The method of radioimmunoassay as described by claim 4, wherein said step of providing an amount of immunoassay sample which provides an amount of liquid which entrains an amount of target particle capable of disossociable capture by said target particle moiety linked to said solid phase substrate of said immunoassay particle comprises the step of an amount of immunoassay sample which provides an amount of liquid which entrains an amount of target particle capable of disossociable capture by said target particle moiety linked to said solid phase substrate of said immunoassay particle, wherein said amount of target particle is selected from group consisting of: an amount of target particle of between about one picogram per mililiter and about three picograms per mililiter, an amount of target particle of between about two picograms per mililiter and about four picograms per mililiter, an amount of target particle of between about three picograms per mililiter and about five picograms per mililiter, an amount of target particle of between about four picograms per mililiter and about six picograms per mililiter, an amount of target particle of between about five picograms per mililiter and about ten picograms per mililiter, an amount of target particle of between about ten picograms per mililiter and about 20 picograms per mililiter, an amount of target particle of between about 20 picograms per mililiter and about 50 picograms per mililiter, an amount of target particle of between about 50 picograms per mililiter and about 100 picograms per mililiter, and an amount of target particle of between about 75 picograms per mililiter and about 150 picograms per mililiter.

6. The method of radioimmunoassay as described by claim 5, further comprising the steps of incubating a combination of said amount of immunoassay sample with said amount of immunoassay particle for an interval of time prior to said step of combining said amount of labeled particle with said amount of immunoassay particle and said amount of immunoassay sample and to generate a volume of not greater than 150 microliters.

7. The method of radioimmunoassay as described by claim 6, further comprising the step of establishing an interval of time to incubate said combination of said amount of immunoassay sample with said amount of immunoassay particle of between about twelve hours and about sixteen hours.

8. The method of radioimmunoassay as described by claim 7, further comprising the step of establishing an interval of time to incubate said combination of said amount of immunoassay sample with said amount of immunoassay particle of between about two hours and about six hours.

9. The method of radioimmunoassay as described by claim 8, wherein said step of providing an amount of labeled particle capable of dissociable capture by said target particle moiety linked to said solid phase substrate of said immunoassay particle further comprises the step of adjusting said amount of labeled particle combined with said immunoassay particle and said amount of immunoassay sample to establish said interval of time of incubation to less than eighteen hours.

10. The method of radioimmunoassay as described by claim 9, wherein said step of providing an amount of labeled particle capable of dissociable capture by said target particle moiety linked to said solid phase substrate of said immunoassay particle further comprises the step of adjusting said amount of labeled particle combined with said immunoassay particle and said amount of immunoassay sample to establish said interval of time of incubation to less than twelve hours.

11. The method of radioimmunoassay as described by claim 10, wherein said step of combining said amount of labeled particle with said amount of immunoassay particle and said amount of immunoassay sample and to generate a volume of not greater than 150 microliters comprises the step of combining said amount of labeled particle with said amount of immunoassay particle and said amount of immunoassay sample and to generate a volume of not greater than 75 microliters.

12. The method of radioimmunoassay as described by claim 11, wherein said step of incubating a combination of said amount of immunoassay sample with said amount of immunoassay particle for an interval of time comprises the step of incubating a combination of said amount of immunoassay sample with said amount of immunoassay particle for an interval of time of not greater than 4 hours.

13. The method of radioimmunoassay as described by claim 11, wherein said step of adjusting said amount of labeled particle combined with said immunoassay particle and said amount of immunoassay sample to establish said interval of time of incubation to less than twelve hours comprises the step of adjusting said amount of labeled particle combined with said immunoassay particle and said amount of immunoassay sample to establish said interval of time of incubation to less than two hours.
